(19) United States
(12) Patent Application Publication
(10) Pub. No.: US 2016/0030022 A1
(43) Pub. Date: Feb. 4, 2016

(54) Optical Biopsy Needle and Endoscope System

(71) Applicant: The General Hospital Corporation, Boston, MA (US)

(72) Inventors: Rahul Sheth, Houston, TX (US); Umar Mahmood, Winchester, MA (US); Anthony Samir, Newton, MA (US)

(21) Appl. No.: 14/788,313

(22) Filed: Jun. 30, 2015

Related U.S. Application Data
(60) Provisional application No. 62/031,331, filed on Jul. 31, 2014.

(51) Int. Cl. A61B 10/04; A61B 1/04; A61B 8/08; A61B 1/07; A61B 1/00; A61B 18/12; A61B 18/20; A61B 1/05; A61B 6/03

(52) U.S. Cl. CPC A61B 10/04; A61B 1/05; A61B 1/043; A61B 8/0841; A61B 6/032; A61B 1/00009; A61B 18/12; A61B 18/20; A61B 1/07; A61B 2010/045; A61B 2018/00577

(57) ABSTRACT

An interventional optical molecular imaging system and method are provided for use with a biopsy introducer needle. The system includes an imaging tool configured to slide coaxially through the biopsy introducer needle toward a tip of the biopsy introducer needle. The system also includes a laser illumination source coupled to the imaging tool, a camera configured to capture images from the imaging tool, and a computer coupled to the camera and the laser illumination source. The computer includes a processor configured to activate the laser illumination source to emit light through a tip of the imaging tool, collect imaging data from the camera after activating the laser illumination source, analyze the imaging data to determine a fluorescence measurement at the tip of the imaging tool, and display an indication of the fluorescence measurement.

### CROSS-REFERENCE TO RELATED APPLICATIONS

This application is based on, claims priority to, and incorporates herein by reference in its entirety, U.S. Provisional Patent Application Ser. No. 62/031,331, filed on Jul. 31, 2014, and entitled “Optical Biopsy Needle and Endoscope System.”

### BACKGROUND

Over the past two decades, improved cross-sectional diagnostic imaging techniques have resulted in earlier detection of focal abnormalities throughout the body. While such non-invasive detection is useful, however, the majority of focal abnormal lesions do not have specific imaging characteristics. For example, lesions smaller than 3 centimeters (cm) often lack specific features to allow reliable noninvasive characterization. Thus, biopsies of focal lesions are often necessary to establish tissue diagnosis of primary or metastatic hepatic malignancy, as well as to perform genotype analysis. Today, percutaneous interventions on focal lesions are among the most commonly performed procedures in Interventional Radiology.

Focal lesion core biopsies are predominantly performed with computer tomography (CT) or ultrasound (US) guidance. However, small lesions can pose several challenges using these techniques. Lesions which are seen on other modalities, such as magnetic resonance imaging (MRI), are sometimes not visible when using CT or US guidance. Alternatively, when the lesion is visible with CT, the biopsy needle introduces beam-hardening artifacts that may render the lesion difficult to see at the critical moment when the needle is in the lesion's vicinity. Furthermore, operator confidence in accurate needle placement using conventional CT or US guidance for biopsies and ablations decreases as target lesions decrease in size. For example, in a study having specialized attending physicians performing liver biopsies, the negative predictive value (NPV) of biopsy for liver lesions less than 3 cm was 72%. Therefore, 28% of cases with liver lesions less than 3 cm having a biopsy negative for malignant cells were ultimately proven to be cancerous. It can be reasoned that in a broader community having generalists performing the liver biopsies, the rate of incorrect benign diagnoses would increase.

In light of the above challenges, biopsies are not considered a “perfect” test. In particular, a negative biopsy does not exclude a cancer diagnosis due to the potential for sampling errors. These sampling errors are often caused by: (1) a targeting failure where the core biopsy needle misses the lesion; (2) a sampling failure where the core biopsy needle intersects the lesion, but does not obtain sufficient malignant tissue; or (3) a tumor visualization failure where the tissue section reviewed by a pathologist does not pass through malignancy in the biopsy specimen. Because of these sampling errors, and a present lack of technology for quickly assessing biopsy adequacy, false negative cancer biopsy is very common in clinical cancer care. These errors result in delayed treatment, repeat biopsy procedures, higher costs, increased patient anxiety, and higher risk of biopsy complications.

Various attempts have been made to reduce the rate of false negative biopsy. For example, some institutions perform fine needle aspiration in addition to core biopsy, and then obtain cytologic “wet reads” of fine needle aspirates while the patient is still on the procedure table. This additional step requires on-site cytology specialist expertise, is not widely available, adds considerable procedure and sedation time, cost, and inconvenience, and does not always predict the final biopsy result. Furthermore, cytologic assessment of fine needle aspirate adequacy does not necessarily imply that core biopsy samples will be adequate for the tumor subtyping and genetic analyses necessary to correctly select personalized cancer therapies.

Therefore, there exists a clinical need for a real-time, accurate method for confirming proper needle position during percutaneous interventions to complement existing image guidance technologies.

### SUMMARY

The systems and method of the present disclosure overcome the above and other drawbacks by providing an optical biopsy needle and endoscope system that complements existing image guidance technologies during percutaneous interventions. More specifically, a real-time, accurate method is provided for confirming proper needle position during tissue biopsy and ablation procedures.

In accordance with one aspect of the disclosure an intraprocedural guidance method is provided. The method includes advancing an introducer needle percutaneously toward the pathologic tissue of the subject and, before reaching the pathologic tissue, obtaining first imaging data of tissue adjacent to a tip of the introducer needle with an imaging tool inserted through the introducer needle. The method also includes further advancing the introducer needle toward the pathologic tissue, obtaining second imaging data of tissue adjacent to the tip of the introducer needle with the imaging tool, and comparing the first imaging data to the second imaging data to determine whether the tip of the introducer needle is at the pathologic tissue. The method further includes generating an output indicating to an operator whether the tip of the introducer needle is at the pathologic tissue.

In accordance with another aspect of the disclosure, an intraprocedural guidance method is provided. The method includes administering an optical imaging agent to a subject, wherein the optical imaging agent targets a pathologic tissue, advancing an introducer needle percutaneously toward the pathologic tissue of the subject, and obtaining target fluorescence imaging data of tissue adjacent to a tip of the introducer needle with an imaging tool inserted through the introducer needle. The method also includes determining whether the tip of the introducer needle is at the pathologic tissue based on a brightness of the target fluorescence imaging data and generating an output indicating to an operator whether the tip of the introducer needle is at the pathologic tissue.

In accordance with yet another aspect of the disclosure, an interventional optical molecular imaging system for use with a biopsy introducer needle is provided. The system includes an imaging tool configured to slide coaxially through the biopsy introducer needle toward a tip of the biopsy introducer needle, where the imaging tool is one of an imaging catheter and a fiber-optic biopsy needle. The system also includes a laser illumination source coupled to the imaging tool, a camera configured to capture images from the imaging tool, and a computer coupled to the camera and the laser illumination source. The computer includes a processor configured to activate the laser illumination source to emit light through a tip of the imaging tool, collect imaging data from the camera after activating the laser illumination source, analyze the imaging data to determine a fluorescence measurement at the tip of the imaging tool, and display an indication of the fluorescence measurement.

The foregoing and other advantages of the invention will appear from the following description. In the description, reference is made to the accompanying drawings that form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

FIG. 1 is a schematic view of an interventional optical molecular imaging system according to one aspect of the present disclosure.

FIG. 2 is a side view of the system of FIG. 1.

FIG. 3 is a partial schematic view of a camera, a filter, and an imaging catheter of the system of FIG. 1.

FIG. 4 is a schematic view of an interventional optical molecular imaging system according to another aspect of the disclosure.

FIG. 5 is a graph illustrating optical imaging agent uptake over time in healthy liver tissue and tumor tissue, and target-to-background ratios of fluorescence measurements over time of the healthy liver tissue and tumor tissue.

FIG. 6 illustrates an intraprocedural guidance method according to one aspect of the disclosure.

FIG. 7 is a graph illustrating tissue temperature over time of healthy liver tissue and a hepatocellular carcinoma.

FIG. 8 illustrates a molecularly targeted ablation method according to one aspect of the disclosure.

### DETAILED DESCRIPTION

The disclosure provides an optical biopsy needle and endoscope system and methods of use that complement existing image guidance technologies during percutaneous interventions. Generally, the system works in conjunction with an administered optical imaging agent that targets specific pathologic tissue. The hand-held system is introduced percutaneously to image tissue at the tip or leading edge of a biopsy introducer needle. If the image indicates presence of the optical imaging agent, the introducer needle is considered properly positioned at the desired pathologic tissue and a biopsy or ablation procedure can be performed. If the image indicates tissue without the optical imaging agent, the introducer needle can be readjusted before the biopsy collection or ablation procedure and further images can be taken to ensure the adjusted introducer needle is properly repositioned. The system's additional guidance for percutaneous sampling of pathologic tissue can help improve accuracy and reduce false-negative biopsy findings, thus reducing the need for repeat biopsy procedures and avoiding needless delays in proper care.

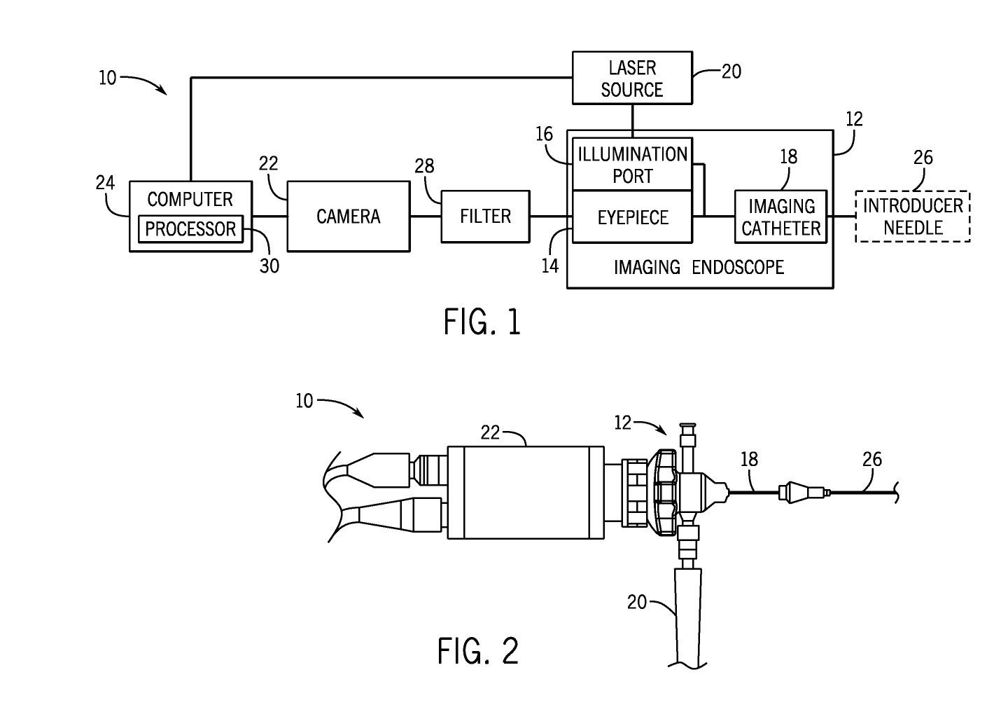

FIG. 1 schematically illustrates an optical biopsy needle and endoscope system 10 according to one aspect of the disclosure. The system 10 can include an imaging endoscope 12 with an eyepiece 14, an illumination port 16, and an imaging catheter 18, a laser or illumination source 20 coupled to the imaging endoscope 12, a camera 22 coupled to the imaging endoscope 12, a computer 24 in communication with the camera 22 and the laser 20, and an introducer needle 26 (for example, from a standard coaxial biopsy system). Generally, the system 10 can be operated by hand and used in conjunction with ultrasound (US) or computed tomography (CT) imaging and an optical imaging agent to help position the introducer needle 26 at the site of pathologic tissue.

In some configurations, the entire system 10 can have a modular design. For example, a variety of imaging endoscopes 12 and lasers 20 can be interchangeable based on the particular application of the system 10. Additionally, the introducer needle 26 can be completely separate and removable from the system 10 so that the system 10 can be reused with different biopsy kits. Also, the camera 22 and the laser 20 can be removably coupled to the computer 24 so that the system 10 can be used with different computers 24 in different procedure rooms.

Different types of imaging endoscopes 12 may be used with the system 10, such as cystoscopes, colonoscopes, pediatric cystoscopes, among others. Generally, the imaging catheter 18 and the introducer needle 26 can be sized so that the imaging catheter 18 can pass coaxially through the introducer needle 26, as shown in FIG. 2, toward a tip of the introducer needle 26. For example, in one configuration, the imaging catheter 18 can be sized to fit through a 17-guage introducer needle 26. Additionally, in some configurations, the imaging catheter 18 and introducer needle 26 are the only components that touch a patient during procedures using the system 10.

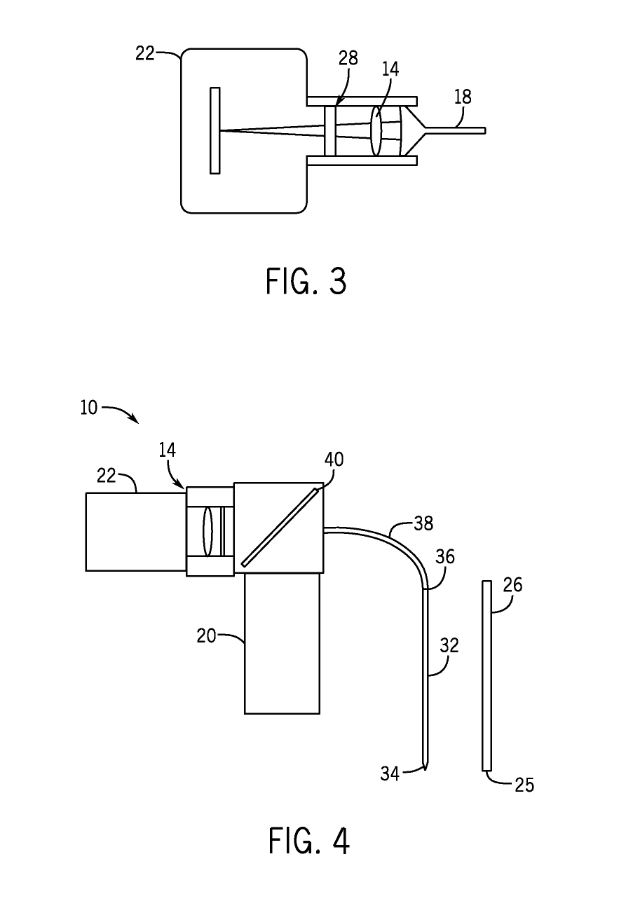

As discussed above, the imaging endoscope 12 can be coupled to the laser 20 and the camera 22 (or other image collection hardware). The laser 20 can be coupled to the illumination port 16, e.g., through a fiber-optic cable, so that activating the laser 20 causes excitation light to be emitted through a tip of the imaging catheter 18. The laser 20 can emit a specific wavelength based on the optical imaging agent. For example, in one configuration, the laser 20 may be a near-infrared (NIR) laser (such as a 450 mW, 785 nm laser) to be used with optical imaging agents that fluoresce under that range. The camera 22 can be configured to capture images from the imaging catheter 18 and, thus, can capture the resulting fluorescence following excitation light emission by the laser 20. More specifically, the camera 22 can be coupled to the eyepiece 14 and, in some configurations, can be a charge-coupled device (CCD) camera, configured to acquire high-temporal and high-spatial resolution images in real time (such as 12-bit images). Additionally, a filter 28, such as a bandpass filter, may be positioned between the camera 22 and the eyepiece 14, as shown in FIGS. 1 and 3. As a result, light collected by the imaging catheter 18 can be filtered to exclude reflected excitation light from the laser 20 from reaching the camera 22. In some configurations, the camera 22 may be optimized to the specific wavelength range as the laser 20 (such as NIR).

The computer 24 can be coupled to the laser 20 and the camera 22 in order to control, for example, camera, laser emission, and image display functions. More specifically, each computer 24 used with the system 10 can include a processor 30 configured to activate the laser 20, collect imaging data from the camera 22, analyze the imaging data, display the imaging data or outputs indicative of the analysis, and/or perform other operations (e.g., by executing a software program stored on the computer 24).

In some configurations, the imaging endoscope 12 may be a stand-alone product usable with various biopsy kits. In other configurations, the imaging endoscope 12 may be integrated into a biopsy or ablation product package. Alternatively, in some configurations, as shown in FIG. 4, a system 10 can include an integrated fiber-optic biopsy needle 32. More specifically, the system 10 can include an eyepiece 14, a laser 20, a camera 22, a computer (not shown), an introducer needle 26, and the biopsy needle 32. The biopsy needle 32 can include an outer metallic cannula with a transparent, diamond-shaped needle tip 34 and can be sized to fit coaxially through the introducer needle 26. At least one fiber-optic bundle (not shown) extends through the length of the biopsy needle 32, taking the place of a separate imaging catheter. In particular, one fiber-optic bundle can extend through the needle 32 for excitation light transmission, and another fiber-optic bundle can extend through the needle 32 for emitted light transmission. A hub 36 of the biopsy needle 32 can be coupled to a liquid light guide 38 further connected to the laser 20 and the camera 22. A dichroic mirror 40 can direct emitted light from the light guide 38 to the camera 22 and excitation light from the laser 20 to the light guide 38 and toward the needle 32.

As discussed above, the system 10 can be used in conjunction with an optical imaging agent to perform optical molecular imaging (OMI) of tissue. Accordingly, the system 10 may be considered an interventional OMI system. OMI encompasses a vast array of imaging modalities, but generally has features such as high spatial resolution, real-time image display, and highly sensitive imaging agents. The optical imaging agent may be selected for a procedure based on the agent's properties relative to a targeted pathologic tissue. More specifically, different optical imaging agents target different regions and/or pathologies, and generally localize at such pathologies with high sensitivity and target-to-background ratios. In other words, an optical imaging agent acts as a “molecular beacon” for pathologic tissue and will concentrate within the pathologic tissue while passing through surrounding healthy tissue. Pathologic tissue may include, but is not limited to, tumors, infectious tissue, and/or inflammatory tissue. Additionally, in some cases, benign and malignant lesions have significantly different imaging agent uptake and, thus, malignant lesions can be differentiated from benign lesions. Additionally, for some tissues, endogenous fluorophores allow for differentiation between normal and abnormal tissue using fluorescence imaging without requiring the administration of an exogenous imaging agent.

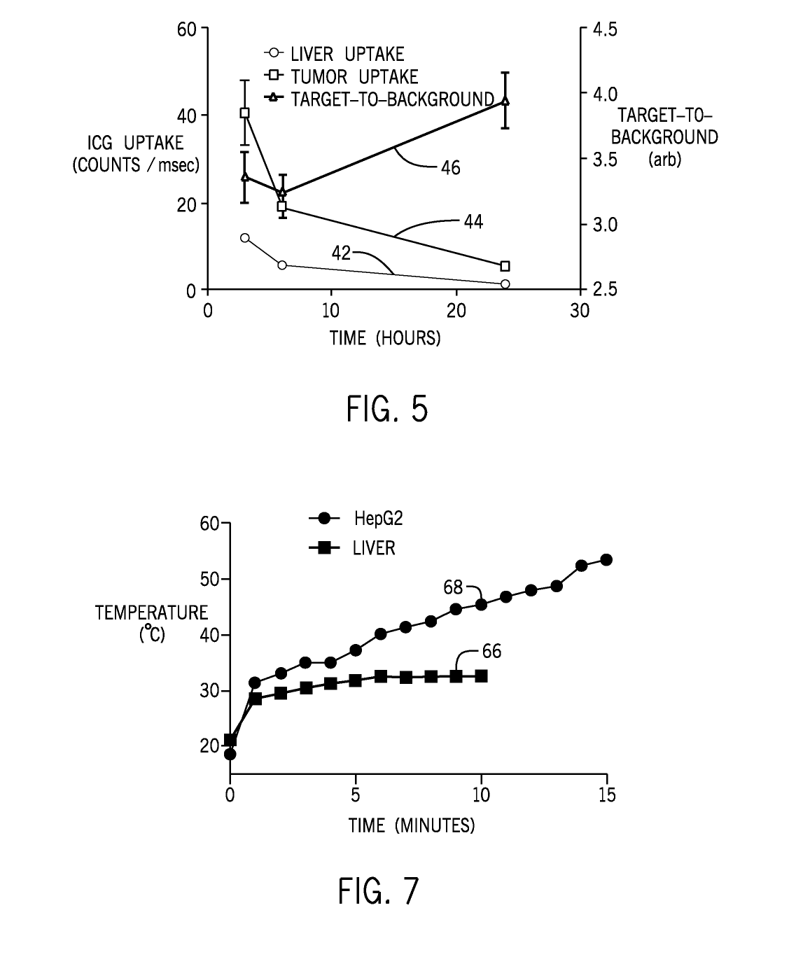

Indocynanine green (ICG) is a clinically approved optical imaging agent that fluoresces in the near-infrared (NIR) range and localizes to hepatocellular carcinoma and hepatic metastatic disease (generally 24-72 hours after injection) with high target-to-background ratios (TBRs). Thus, upon administration of ICG to a patient with either pathology, such pathologic tissue will fluoresce during OMI with NIR light while surrounding healthy tissue will not fluoresce (or will minimally fluoresce compared to the pathologic tissue), providing a high target-to-background, or pathologic tissue-to-healthy tissue, ratio. In particular, FIG. 5 illustrates ICG uptake over time of healthy liver tissue 42 and a liver tumor 44, as well as TBRs 46 comparing fluorescence of the healthy tissue and the tumor tissue over time. As shown in FIG. 5, there is markedly less ICG uptake in healthy tissue compared to tumor tissue, resulting in high TBRs. Thus, by imaging tissues percutaneously with the system 10, an interventionalist can observe “bright spots” within collected images, indicating pathologies specific to the optical imaging agent.

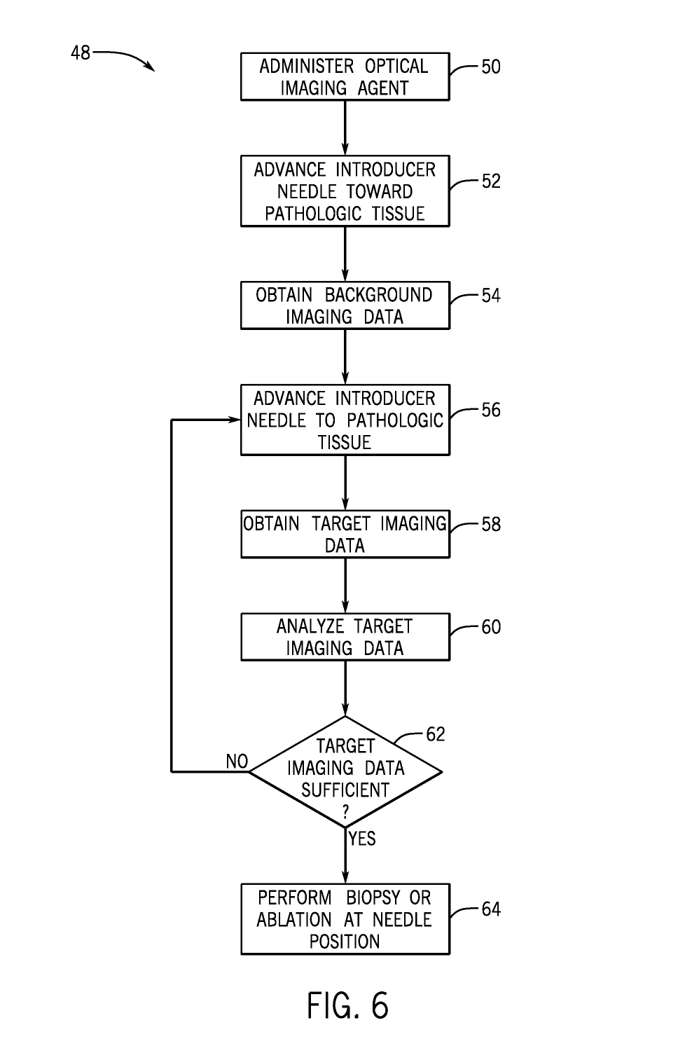

Accordingly, FIG. 6 illustrates an intraprocedural guidance method 48, according to one aspect of the disclosure, using the system 10 for biopsy or ablation procedures. The method 48 may be performed with either imaging tool of the system 10 (i.e., the imaging catheter 18 or the fiber-optic biopsy needle 32). Generally, the method 48 includes administering an optical imaging agent to a subject (process block 50) and introducing the introducer needle 26 percutaneously toward the pathologic tissue (process block 52). The method 48 also includes obtaining background imaging data while the introducer needle 26 is still in an area of healthy tissue (process block 54) then further advancing the introducer needle 26 to the pathologic tissue (process block 56). Once at the pathologic tissue, the method 48 includes obtaining target imaging data (process block 58) and analyzing the target imaging data (process block 60) to determine whether such data is sufficient (process block 62). If the target imaging data is insufficient, process blocks 56-62 are repeated. If the target imaging data is sufficient, a biopsy or ablation procedure is performed at the current introducer needle position (process block 64).

More specifically, process block 50 includes administering an optical imaging agent to a subject and waiting for the optical imaging agent to reach the target pathologic tissue. In some configurations, this waiting period may take minutes, hours, or days. Additionally, in configurations where pathologic tissue fluoresces differently than normal tissue without the addition of an optical imaging agent, this step may be eliminated. At process block 52, the introducer needle 26 is introduced percutaneously toward the pathologic tissue by an operator using US or CT guidance.

Before reaching the pathologic tissue (that is, while in an area of healthy tissue adjacent to the pathologic tissue) background imaging data is obtained (process block 54). Using the system 10 of FIG. 1, a conventional biopsy needle, or inner stylet (not shown), is removed from the introducer needle 26 and the imaging catheter 18 is inserted through the introducer needle 26. Using the system 10 of FIG. 4, the fiber-optic biopsy needle 24 can remain within the introducer needle 26. The computer 24 then operates the laser 20 and the camera 22 to collect the imaging data of tissue at a tip 25 of the introducer needle 24. In particular, an operator can provide input to the computer 24, directing the computer 24 to initiate image data collection. In response to this input, the computer 24 operates the laser 20 to emit excitation light toward the tissue at the introducer needle tip 25 and then operates the camera 22 to collect image data of light emitted from the tissue at the introducer needle tip 25 in response to the excitation light.

In some configurations, the background imaging data can include raw imaging data from the camera 22, or processed imaging data, such as a fluorescence measurement. For example, fluorescence intensity measurements are measured by drawing a region of interest within a collected circular projected 12-bit image and calculating a mean pixel intensity of the region of interest (e.g., using an image analysis software package). Additionally, the fluorescence intensity can be normalized by dividing the mean pixel intensity by the exposure time of the image. Normalized intensities can allow comparison between images with varying exposure times. Also, in some configurations, image processing techniques may be applied to the background imaging data, including scatter reduction techniques (such as angular ‘memory-effect’ for speckle correlations and an autocorrelation method). In some configurations, the computer 24 may display an image of the tissue, a numerical fluorescence measurement, and/or a status symbol indicating that the measurement was taken. Once the background imaging data is collected, using the system 10 of FIG. 1, the imaging catheter 18 is removed from the introducer needle 26 and the stylet reinserted.

After collecting background imaging data, the introducer needle 26 is then advanced to reach the pathologic tissue using US or CT guidance (process block 56). The stylet and the imaging catheter 18 are again interchanged, if using the system 10 of FIG. 1, and target imaging data is obtained of the tissue at the tip of the introducer needle 26 (process block 58). The target imaging data may be obtained and/or processed in a similar manner as the background imaging data. For example, the target imaging data may be a fluorescence measurement. In some configurations, the computer 24 may display an image, a numerical fluorescence measurement, and/or a status symbol indicating that the measurement was taken.

At process block 60, the target imaging data is analyzed by the computer 24 (e.g., via a specific software program) and/or by an operator (such as an interventional radiologist or surgeon). The purpose of the analysis at process block 60 is to determine whether the target imaging data is sufficient (process block 62). Sufficient target imaging data can indicate presence of the optical imaging agent and, more specifically, introducer needle tip position at the pathologic tissue. In one example, the introducer needle tip 25 may be considered positioned at the pathologic tissue if the introducer needle tip 25 is within the pathologic tissue or within about 10 millimeters (mm) to about 1 centimeter (cm) of the pathologic tissue.

Analysis at process block 60 can include visual analysis of images and/or numerical analysis of fluorescence measurement data. Furthermore, analysis may include analyzing only the target imaging data (e.g., observing brightness or fluorescence intensity of the target imaging data) or using the background imaging data as a baseline and comparing target imaging data to background imaging data. For example, TBRs can be calculated by dividing the mean fluorescence intensity of the target imaging data by the mean fluorescence intensity of the background imaging data. Also, in some configurations, analysis may include comparing target imaging data, such as fluorescence intensity, to stored baseline or threshold intensities correlated with known histologic diagnoses. In some configurations, the computer 24 may display one or more outputs of the analysis, such as side-by-side images (i.e., background and target tissues), numerical fluorescence measurements, TBRs, status symbols indicating that the target measurement is sufficient or insufficient, and/or potential histologies.

Process blocks 56-62 may be repeated until target imaging data is sufficient, i.e., until the output indicates that the introducer needle tip 25 is at the pathologic tissue. Once target imaging data is sufficient, a biopsy or ablation procedure may be performed (process block 64). More specifically, a biopsy sample maybe collected using the stylet or the fiber-optic biopsy needle 32, and the method 48 (or at least process blocks 56-64) may be repeated to obtain multiple biopsy samples. Alternatively, an ablation probe, such as a microwave antenna, can be inserted through the introducer needle 26 to perform, for example, microwave or radiofrequency ablation. The method 48 (or at least process blocks 56-64) may be repeated to ensure the pathologic tissue, in its entirety, is sufficiently burned via the ablation procedure. Additionally, in some configurations, process block 62 can include performing a fine needle aspiration procedure.

With respect to ablation procedures, the system 10 and the method 48 can help better define pathological tissue margins. More specifically, the burning process makes such margins less clear under conventional imaging, such as CT or US, but the system 10 can provide images with sharp margins of fluorescence intensity at the healthy-pathologic tissue interface. Furthermore, the method 48 may help ensure that all pathological tissue has been sufficiently ablated in real time, since ablated tissue will no longer fluoresce under excitation light. Thus, the system 10 and method 48 can ensure that there is no residual pathologic tissue left untreated.

In some configurations, the system 10 can be used to perform laser illumination thermal ablation, without requiring an additional ablation probe. More specifically, most of the excitation light emitted by the laser 20 and absorbed by an optical imaging agent is converted to heat. For example, while ICG fluoresces under NIR excitation light, about 95% of the excitation light absorbed by ICG is converted to heat. As a result, excitation light can be used to target areas containing an optical imaging agent for thermal ablation. Because the optical imaging agent does not remain in surrounding healthy tissue, excitation light has little heating effect on such healthy tissue. In a recent study, after administration of ICG, NIR excitation light was applied to a healthy liver tissue and a hepatocellular carcinoma (Hep G2). FIG. 7 illustrates tissue temperature over time of both the healthy tissue 66 and the carcinoma 68. As shown in FIG. 7, the excitation light “activated” the optical imaging agent to heat the tumor tissue (which reached about 50 degrees Celsius—high enough to kill the tumor cells), but had little effect on surrounding the healthy tissue (which remained near normal body temperature).

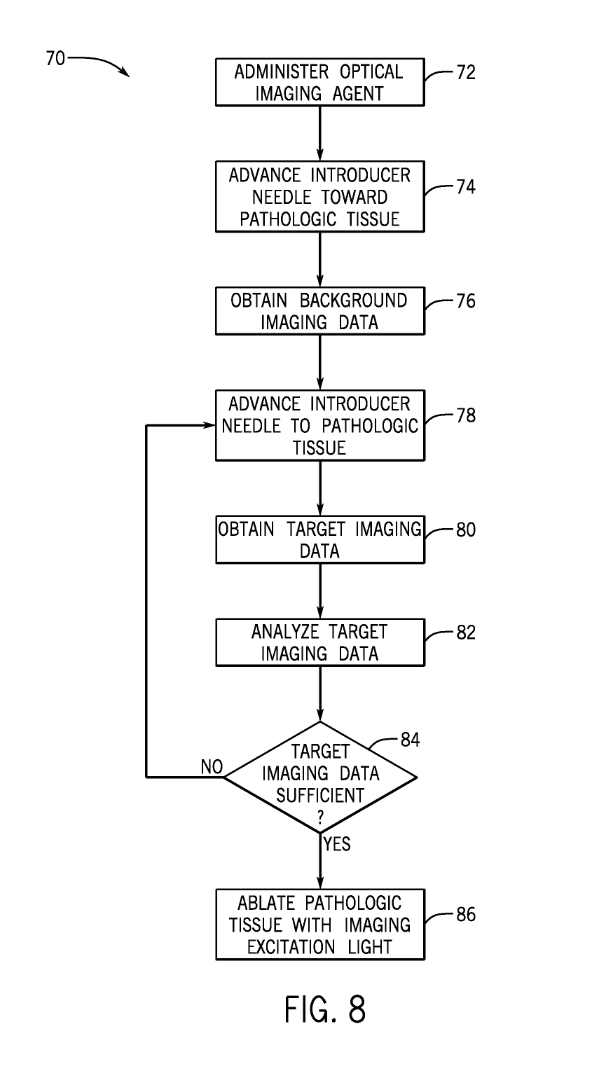

FIG. 8 thus illustrates a molecularly targeted ablation method 70, for use with the system 10. Generally, the method 70 includes the same initial steps as the method 48 of FIG. 6: administering an optical imaging agent to a subject (process block 72); introducing the introducer needle 26 percutaneously toward the pathologic tissue (process block 74); obtaining background imaging data while the introducer needle 26 is still in an area of healthy tissue (process block 76); further advancing the introducer needle 26 to the pathologic tissue (process block 78); once at the pathologic tissue, obtaining target imaging data (process block 80); analyzing the target imaging data (process block 82) to determine whether such data is sufficient (process block 84); and if the target imaging data is insufficient, repeating process blocks 78-84. If the target imaging data is sufficient, the method 70 further includes using the laser 20, as controlled by the computer 24, and the imaging endoscope 18 or the fiber-optic biopsy needle 32 to shine excitation light on the pathologic tissue for a time period (process block 86). An operator may then repeat steps 78-86 until the pathologic tissue, in its entirety, is ablated.

In light of the above, aspects of the disclosure provide a system and methods for image-guided percutaneous interventions. The present interventional OMI system 10 is compatible with a standard coaxial biopsy system and, thus is consistent with the minimally invasive nature of interventional radiology procedures. By using the system 10 in conjunction with an optical imaging agent, tissue at the introducer needle tip 25 can be interrogated for pathology prior obtaining a core specimen. Thus, pathologic tissue can be readily differentiated from healthy tissue in a minimally invasive manner. Also, real-time feedback from the imaging data during biopsy or ablation can provide a radiologist with information that directs sampling location, procedure end points, and/or drug selection.

The system 10 may reduce overall procedure time by improving operator confidence in biopsy needle positioning. Moreover, the improved operator confidence can enable sampling smaller pathologies, thereby allowing physicians to provide a diagnosis and appropriate management plan to patients earlier in the course of disease. Furthermore, given the molecular specificity of optical imaging agents, a quantitative assessment of the degree of molecular agent uptake could provide an “in vivo histology” diagnosis without requiring full tissue samples, which may be helpful for pathologies in locations where obtaining a core biopsy specimen may be dangerous.

Additionally, in some configurations, the system 10 can be used to detect pathologic tissue during surgical resection, as an alternative to or in conjunction with standard visual inspection and intraoperative US. Also, in some configurations, the system 10 and methods of the present disclosure can be used to image neoplasia endoscopically. More specifically, the system 10 can be integrated into conventional endoscopic systems to enable simultaneously imaging fluorescence and white light in vivo. The system 10 can provide a fluorescence image, enabling observation of pathologic lesions that would be difficult to visualize by standard white light images alone. In one example, the system 10 may be used to detect colonic adenomas in vivo following intravenous administration of an optical imaging agent, such as ICG.

The present invention has been described in terms of one or more preferred embodiments, and it should be appreciated that many equivalents, alternatives, variations, and modifications, aside from those expressly stated, are possible and within the scope of the invention.

1. An intraprocedural guidance method including steps comprising:
a) advancing an introducer needle percutaneously toward the pathologic tissue of the subject;
b) before reaching the pathologic tissue, obtaining first imaging data of tissue adjacent to a tip of the introducer needle with an imaging tool inserted through the introducer needle;
c) further advancing the introducer needle toward the pathologic tissue;
d) obtaining second imaging data of tissue adjacent to the tip of the introducer needle with the imaging tool;
e) comparing the first imaging data to the second imaging data to determine whether the tip of the introducer needle is at the pathologic tissue; and
f) generating an output indicating to an operator whether the tip of the introducer needle is at the pathologic tissue.

2. The intraprocedural guidance method of claim 1, wherein the imaging tool is a fiber-optic biopsy needle configured to emit excitation light, collect emitted light, and obtain a tissue biopsy sample.

3. The intraprocedural guidance method of claim 1 and further comprising repeating steps c) through f) until the output indicates that the tip of the introducer needle is at the pathologic tissue.

4. The intraprocedural guidance method of claim 3 and further comprising one of obtaining a biopsy of the pathologic tissue and ablating the pathologic tissue when the tip of the introducer needle is at the pathologic tissue.

5. The intraprocedural guidance method of claim 4 and further comprising administering an optical imaging agent to a subject, wherein the optical imaging agent targets a pathologic tissue, wherein ablating the pathologic tissue includes illuminating the optical imaging agent within the pathologic tissue using the imaging tool.

6. The intraprocedural guidance method of claim 1, wherein the first imaging data and the second imaging data are fluorescence measurements.

7. The intraprocedural guidance method of claim 1, wherein the output is one of a displayed image of tissue, displayed numerical data, and a displayed symbol.

8. The intraprocedural guidance method of claim 7, wherein the displayed numerical data is one of fluorescence intensity and a target-to-background ratio.

9. The intraprocedural guidance method of claim 1 and further comprising using one of ultrasound and computed tomography guidance to advance the introducer needle.

10. An intraprocedural guidance method including steps comprising:
a) administering an optical imaging agent to a subject, wherein the optical imaging agent targets a pathologic tissue;
b) advancing an introducer needle percutaneously toward the pathologic tissue of the subject;
c) obtaining target fluorescence imaging data of tissue adjacent to a tip of the introducer needle with an imaging tool inserted through the introducer needle;
d) determining whether the tip of the introducer needle is at the pathologic tissue based on a brightness of the target fluorescence imaging data; and
e) generating an output indicating to an operator whether the tip of the introducer needle is at the pathologic tissue.

11. The intraprocedural guidance method of claim 10, wherein the step of determining whether the tip of the introducer needle is at the pathologic tissue includes determining whether the brightness indicates presence of the optical imaging agent at the tip of the introducer needle.

12. The intraprocedural guidance method of claim 10 and further comprising using one of ultrasound and computed tomography guidance to advance the introducer needle.

13. The intraprocedural guidance method of claim 10, wherein the imaging tool is one of an imaging catheter and a fiber-optic biopsy needle, wherein the fiber-optic biopsy needle is configured to emit excitation light, collect emitted light, and obtain a tissue biopsy sample.

14. The intraprocedural guidance method of claim 10, wherein when the output indicates that the tip of the introducer needle is at the pathologic tissue, further comprising introducing a biopsy needle through the introducer needle and obtaining a biopsy sample of the pathologic tissue at the tip of the introducer needle.

15. The intraprocedural guidance method of claim 10, wherein when the output indicates that the tip of the introducer needle is at the pathologic tissue, further comprising introducing an ablation probe through the introducer needle and ablating the pathologic tissue at the tip of the introducer needle.

16. The intraprocedural guidance method of claim 10, wherein when the output indicates that the tip of the introducer needle is at the pathologic tissue, further comprising ablating the pathologic tissue at the tip of the introducer needle by illuminating the optical imaging agent within the pathologic tissue using the imaging tool.

17. An interventional optical molecular imaging system for use with a biopsy introducer needle, the system comprising:
an imaging tool configured to slide coaxially through the biopsy introducer needle toward a tip of the biopsy introducer needle, the imaging tool including one of an imaging catheter and a fiber-optic biopsy needle;
a laser illumination source coupled to the imaging tool;
a camera configured to capture images from the imaging tool; and
a computer coupled to the camera and the laser illumination source, the computer including a processor configured to:
activate the laser illumination source to emit light through a tip of the imaging tool,
collect imaging data from the camera after activating the laser illumination source,
analyze the imaging data to determine a fluorescence measurement at the tip of the imaging tool, and
display an indication of the fluorescence measurement.

18. The system of claim 17 wherein the imaging data includes baseline tissue imaging data and target tissue imaging data, and the processor is further configured to:
determine a baseline fluorescence measurement from the baseline tissue imaging data and a target fluorescence measurement from the target tissue imaging data,
compare the baseline fluorescence measurement and the target fluorescence measurement, and
display an indication of the comparison.

19. The system of claim 18, wherein the indication of the comparison is a numerical target-to-background ratio.

20. The system of claim 17 and further comprising a filter positioned between the camera and the imaging tool.
